(19) United States
(12) Patent Application Publication
(10) Pub. No.: US 2007/0009474 A1
(43) Pub. Date: Jan. 11, 2007

(54) Personal care compositions and methods for the beautification of mammalian skin and hair

(75) Inventors: Sancai Xie, West Chester, OH (US); Kotikanyadanam Sreekrishna, Cincinnati, OH (US); Abby Ballard Newland, Lawrenceburg, IN (US); Charles Carson Bascom, Hamilton, OH (US); Joseph Robert Kaczvinsky JR., Cincinnati, OH (US); Keren Marie Lammers, North Bend, OH (US); Kristina Emma Inge Vanoosthuyze, Horsell Woking (GB)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/482,314

(22) Filed: Jul. 7, 2006

Related U.S. Application Data
(60) Provisional application No. 60/697,819, filed on Jul. 8, 2005.

(51) Int. Cl. A61K 8/97; A61K 31/522

(52) U.S. Cl. 424/74; 514/263.31; 514/263.32

(57) ABSTRACT

Personal care composition comprising from about 0.05% to about 5% of at least one aquaporin-stimulating compound selected from the group consisting of xanthine, caffeine; 2-amino-6-methyl-mercaptopurine; 1-methyl xanthine; 2-aminopurine; theophylline; theobromine; adenine; adenosine; kinetin; p-chlorophenoxyacetic acid; 2,4-dichlorophenoxyacetic acid; indole-3-butyric acid; indole-3-acetic acid methyl ester; beta-naphthoxyacetic acid; 2,3,5-triiodobenzoic acid; adenine hemisulfate; n-benzyl-9-(2-tetrahydropyranyl)adenine; 1,3-diphenylurea; 1-phenyl-3-(1,2,3-thiadiazol-5-yl)urea; zeatin; indole-3-acetic acid; 6-benzylaminopurine; alpha-napthaleneacetic acid; 6-2-furoylaminopurine; green tea extract; white tea extract; menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor and combinations thereof; an additional ingredient selected from the group consisting of niacinamide, glycerin and mixtures thereof, and a dermatologically-acceptable carrier.

### CROSS REFERENCE TO RELATED APPLICATION

This application claims the benefit of U.S. Provisional Application No. 60/697,819, filed Jul. 8, 2005.

### FIELD OF THE INVENTION

The present invention relates to personal care compositions comprising aquaporin-stimulating compounds, and methods of use thereof.

### BACKGROUND OF THE INVENTION

A number of personal care products currently are available to consumers, which are directed toward improving dry skin. The outermost layer of the skin, the stratum corneum, receives water by being brought into direct contact with water or via diffusion from the underlying epidermis. The diffusion process is controlled by the water content of the skin as well as the concentration gradient. In a very dry environment, evaporative water loss from the stratum corneum can be significant and often exceeds the rate of replacement by diffusion. Compositions containing humectants, occlusive or semi-occlusive substances, and/or materials that improve barrier function can inhibit or retard evaporative water loss, but have the disadvantage of only minimally affecting diffusion.

Aquaporins are a class of membrane proteins within mammalian skin that regulate the transport of water, glycerol and other solutes across the plasma membrane. Without being limited by theory, two major aquaporin membrane proteins, AQP-3 and AQP-9, are expressed in skin. AQP-3 is a transporter protein in the plasma membrane of keratinocytes, which transports water and glycerol into the vascular-free epidermis from the dermis. When AQP-3 gene is inactivated, multiple symptoms of damaged skin, such as lower water content, leaky skin barrier, delayed wound healing and impaired skin elasticity, are observed. It is believed that an increase in the expression of AQP-3 in skin improves skin hydration, thus minimizing the visual signs of dry or photo-damaged skin, and delivering benefits in skin moisturization, appearance, tone, texture and firmness. There exists a need, therefore, to identify compounds that stimulate aquaporin membrane proteins.

### SUMMARY OF THE INVENTION

The present invention meets the aforementioned need. Applicants identify herein active ingredients useful for stimulating aquaporin membrane proteins, and compositions useful for providing one or more benefits to the mammalian keratinous tissue to which they are applied.

The following represent some non-limiting embodiments of the present invention.

In one embodiment, a composition is provided comprising an effective amount of at least one aquaporin-stimulating compound, an additional ingredient selected from the group consisting of from niacinamide, glycerin and mixtures thereof, and a dermatologically-acceptable carrier.

In yet another embodiment, a method for regulating the condition of mammalian keratinous tissue is provided, comprising the step of applying to a portion of mammalian keratinous tissue in need of regulation an effective amount of the personal care composition of the present invention.

In yet another embodiment, a kit for regulating the condition of mammalian skin is provided, comprising at least one composition as described herein.

### DETAILED DESCRIPTION OF THE INVENTION

In all embodiments of the present invention, all percentages are by weight of the total composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “personal care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

“Keratinous tissue,” as used herein, refers to keratin-containing layers disposed as the outermost protective covering of mammals which includes, but is not limited to, skin, hair, nails, cuticles, etc.

“Topical application”, as used herein, means to apply or spread the compositions of the present invention onto the surface of the keratinous tissue.

“Dermatologically acceptable,” as used herein, means that the compositions or components described are suitable for use in contact with human keratinous tissue without undue toxicity, incompatibility, instability, allergic response, and the like.

“Effective amount” as used herein means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

Herein, “delivery enhancement device” means any device that increases the amount of active ingredient applied to and/or into the skin relative to the amount of active ingredient that is delivered without using the device.

Herein, “regulating skin condition” means improving skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, and graying hair.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

Herein “kit” means a packaging unit comprising at least one composition described herein.

I. Aquaporin-Stimulating Compounds

The composition of the present invention may comprise from about 0.05% to about 5%, alternatively from about 0.1% to about 3%, and alternatively from about 0.1% to about 1.5%, of at least one aquaporin-stimulating compound, including but not limited to xanthine, caffeine; 2-amino-6-methyl-mercaptopurine; 1-methyl xanthine; 2-aminopurine; theophylline; theobromine; adenine; adenosine; kinetin; p-chlorophenoxyacetic acid; 2,4-dichlorophenoxyacetic acid; indole-3-butyric acid; indole-3-acetic acid methyl ester; beta-naphthoxyacetic acid; 2,3,5-triiodobenzoic acid; adenine hemisulfate; n-benzyl-9-(2-tetrahydropyranyl)adenine; 1,3-diphenylurea; 1-phenyl-3-(1,2,3-thiadiazol-5-yl)urea; zeatin; indole-3-acetic acid; 6-benzylaminopurine; alpha-napthaleneacetic acid; 6-2-furoylaminopurine; Pluronic L43™; Tetronic 908™; tetronic 1107; green tea extract; white tea extract; menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor and combinations thereof; an effective amount of niacinamide; and the balance carriers and adjuncts. Without wishing to be bound by theory, it is believed that compounds that stimulate the aquaporin membrane protein in the epidermis can provide benefits to the mammalian skin to which they are applied including but not limited to: enhanced barrier function, improved skin and hair hydration; increased firmness of skin; and reduction of fine lines and wrinkles.

The composition further may comprise an additional ingredient selected from the group consisting of from niacinamide, glycerin and mixtures thereof. Without wishing to be bound by theory, it is believed that the use of niacinamide in conjunction with at least one aquaporin-stimulating compound provides enhanced beautification benefits to mammalian skin by stimulating transport of water or glycerol within the epidermis, resulting in increased skin hydration, enhanced production of water binding molecules (e.g., hyaluronic acid) in skin, improved epidermal stratification and barrier formation; skin firming; and reduction in visible flakes, lines and wrinkles. It is further believed that the use of glycerin in conjunction with one or more aquaporin-stimulating compound results in enhanced beautification benefits to the mammalian skin to which it is applied by enhancing production of aquaporins in the epidermis (and the benefits thereof), improving skin hydration; improving barrier function and skin firming; increasing stratum corneum hydration; and reducing visible flakes, lines and wrinkles.

In one embodiment of the present invention, the composition may comprise from about 1% to about 8%, alternatively from about 2% to about 5%, and alternatively from about 3% to about 5%, of niacinamide. In another embodiment of the present invention, the composition may comprise from about 9% to about 30%, alternatively from about 10% to about 25%, alternatively from about 10% to about 20%, and alternatively from about 5% to about 9% of glycerin. In another embodiment, the composition may comprise glycerin in a weight percent ratio with at least one aquaporin-stimulating compound of from about 1 to about 6.666, preferably from about 1 to about 4, more preferably from about 1 to about 2.

In yet another embodiment, the composition comprise at least one aquaporin-stimulating compound, glycerin and niacinamide present in a weight percent ratio of from about 1 to about 6.66 to about 3.33, respectively; alternatively from about 1 to about 4 to about 2, respectively; and alternatively from about 1 to about 2 to about 1, respectively.

II. Skin Care Actives

The present invention may include additional hair and/or skin care actives, collectively referred to as “skin care actives,” selected from the group consisting of sugar amines, vitamin B₃, retinoids, peptides, dialkanoyl hydroxyproline, hexamidine, salicylic acid, phytosterol, sunscreen actives, water soluble vitamins, oil-soluble vitamins, their derivatives, their precursors, and combinations thereof.

1. Sugar Amines (Amino Sugars)

The composition of the present invention may comprise a sugar amine, which are also known as amino sugars. The sugar amine compounds useful in the present invention are described in PCT Publication WO 02/076423 and U.S. Pat. No. 6,159,485.

In one embodiment, the composition may contain from about 0.01% to about 15%, alternatively from about 0.1% to about 10%, and alternatively from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Non-limiting examples of sugar amines useful herein include glucosamine, N-acetyl glucosamine, mannosamine, N-acetyl mannosamine, galactosamine, N-acetyl galactosamine, their isomers, salts (e.g., HCl salt) and derivatives. In one embodiment, the sugar amine is glucosamine, alternatively D-glucosamine and alternatively N-acetyl-D-glucosamine.

2. Vitamin B₃

The composition of the present invention may comprise a vitamin B₃ compound. Vitamin B₃ compounds are particularly useful for regulating skin condition as described in U.S. Pat. No. 5,939,082. In one embodiment, the composition may comprise from about 0.01% to about 50%, alternatively from about 0.1% to about 20%, alternatively from about 0.5% to about 10%, alternatively from about 1% to about 7%, and alternatively from about 2% to about 5%, of the vitamin B₃ compound. Non-limiting examples of derivatives of the vitamin B₃ compounds include nicotinic acid esters, including non-vasodilating esters of nicotinic acid (e.g., tocopheryl nicotinate, myristyl nicotinate).

3. Retinoids

The composition of the present invention may comprise a retinoid, such that the resultant composition is suitable for regulating visible and/or tactile discontinuities in skin, for example, for regulating signs of skin aging. In one embodiment, the composition may comprise from about 0.001% to about 10%, alternatively from about 0.005% to about 2%, alternatively from about 0.01% to about 1%, and alternatively from about 0.01% to about 0.5%, by weight of the composition, of the retinoid. The optimum concentration used in a composition will depend on the specific retinoid selected since their potency may vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid may be selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. In one embodiment, the retinoid is one other than retinoic acid. In one embodiment, the retinoid is selected from the group consisting of retinol, retinyl palmitate, retinyl acetate, retinyl propionate, retinal and combinations thereof. Alternatively, the retinoid is retinyl propionate, present in an amount of from about 0.1% to about 0.3%.

4. Peptides

The composition of the present invention may comprise a peptide, including but not limited to, di-, tri-, tetra-, penta-, and hexa-peptides and derivatives thereof. The composition may comprise from about 1×10⁻⁶% to about 20%, alternatively from about 1×10⁻⁶% to about 10%, and alternatively from about 1×10⁻⁵% to about 5%, and alternatively from about 0.001% to about 1%.

As used herein, “peptide” refers to peptides containing ten or fewer amino acids and their derivatives, isomers, and complexes with other species such as metal ions, including but not limited to copper, zinc, manganese, magnesium, etc. As used herein, peptide refers to both naturally occurring and synthesized peptides. Also useful herein are naturally occurring and commercially available compositions that contain peptides. for example, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®). One example of a commercially available tripeptide derivative-containing composition is Biopeptide CL®, which contains 100 ppm of palmitoyl-gly-his-lys and is commercially available from Sederma.

5. Phytosterols

The compositions of the present invention may comprise one or more phytosterols selected from the group consisting of β-sitosterol, campesterol, brassicasterol, Δ5-avennasterol, lupenol, α-spinasterol, stigmasterol, their derivatives, analogs, and combinations thereof. In one embodiment, the phytosterol is selected from the group consisting of β-sitosterol, campesterol, brassicasterol, stigmasterol, their derivatives, and combinations thereof. In one embodiment, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. In one embodiment, the phytosterols are free sterols. As used herein, “phytosterol” includes isomers, derivatives and tautomers of such and are commercially available from Aldrich Chemical Company, Sigma Chemical Company, and Cognis.

The composition of the present invention may comprise from about 0.0001% to about 25%, alternatively from about 0.001% to about 15%, alternatively from about 0.01% to about 10%, alternatively from about 0.1% to about 5%, and alternatively from about 0.2% to about 2% of the phytosterol.

5. Hexamidine

The composition of the present invention may comprise hexamidine. “Hexamidine,” as used herein, includes isomers, tautomers, salts and derivatives of hexamidine, including but not limited to organic acids and mineral acids, for example sulfonic acid, carboxylic acid, etc. A technical name for the hexamidine of the present invention is 4,4′-(hexamethylenedioxy) dibenzenecarboximidamide. Dermatologically acceptable salts include alkali metal salts, such as sodium and potassium; alkaline earth metal salts, such as calcium and magnesium; non-toxic heavy metal salts; and ammonium and trialkylammonium salts such as trimethylammonium and triethylammonium. Alternatively, the hexamidine is hexamidine isethionate, which is commercially available under the tradename ELASTAB® HP100 from Laboratoires Serobiologiques (Pulnoy, France).

The composition of the present invention may comprise from about 0.0001% to about 25%, alternatively from about 0.001% to about 10%, alternatively from about 0.01% to about 5%, and alternatively from about 0.02% to about 2.5% by weight of the composition.

7. Dialkanoyl Hydroxyproline Compounds

The compositions of the present invention may comprise one or more dialkanoyl hydroxyproline compounds and salts and derivatives thereof. The composition may comprise from about 0.01% to about 10%, alternatively from about 0.01% to about 5%, alternatively from about 0.1% to about 2% of a dialkanoyl hydroxyproline compound.

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. In one embodiment the dipalmitoyl hydroxyproline is the triethanolamine salt of dipalmitoyl hydroxyproline.

8. Salicylic Acid Compound

The composition of the present invention may comprise a salicylic acid compound, and esters, salts, and derivatives thereof. In the compositions of the present invention, the salicylic acid compound may comprise from about 0.0001% to about 25%, alternatively from about 0.001% to about 15 alternatively from about 0.01% to about 10%, alternatively from about 0.1% to about 5%, and alternatively from about 0.2% to about 2%, of salicylic acid.

9. N-acyl Amino Acid Compound

The composition of the present invention may comprise one or more N-acyl amino acid compounds. In one embodiment, the N-acyl amino acid compound is selected from the group consisting of N-acyl Phenylalanine, N-acyl Tyrosine, their isomers, their salts, and derivatives thereof. The amino acid can be the D or L isomer or a mixture thereof.

Particularly useful as a topical skin tone evening (lightening or pigmentation reduction) cosmetic agent is N-undecylenoyl-L-phenylalanine, commercially available under the tradename Sepiwhite® from SEPPIC. This agent belongs to the broad class of N-acyl Phenylalanine derivatives, with its acyl group being a C11 mono-unsaturated fatty acid moiety and the amino acid being the L-isomer of phenylalanine.

The composition of the present invention may comprise from about 0.0001% to about 25%, alternatively from about 0.001% to about 10%, alternatively from about 0.01% to about 5%, and alternatively from about 0.02% to about 2.5% of the N-acyl amino ac id

10. Sunscreen Actives

The composition of the present invention may comprise one or more sunscreen actives (or sunscreen agents) and/or ultraviolet light absorbers, and may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-11, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxycinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimid-azole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof.

In one embodiment, the composition may comprise from about 1% to about 30%, and alternatively from about 2% to about 20%, of the sunscreen active and/or ultraviolet light absorber. Exact amounts will vary depending upon the chosen sunscreen active and/or ultraviolet light absorber and the desired Sun Protection Factor (SPF) and spectrum of protection (e.g. UV-A and/or UV-B), and are within the knowledge and judgment of one of skill in the art.

11. Water-Soluble Vitamins

The compositions of the present invention may comprise one or more water-soluble vitamins. Examples of water-soluble vitamins include, but are not limited to, water-soluble versions of vitamin B (such as vitamin B5 and vitamin B6), vitamin B derivatives, vitamin C (such as ascorbyl glucoside), vitamin C derivatives (such as magnesium ascorbyl phosphate, sodium ascorbyl phosphate, and ascorbyl palmitate), vitamin K, vitamin K derivatives, pro-vitamins thereof, such as panthenol and mixtures thereof. The composition may comprise from about 0.0001% to about 50%, alternatively from about 0.001% to about 10%, alternatively from about 0.01% to about 8%, and alternatively from about 0.1% to about 5%, of the vitamin compound.

12. Oil-Soluble Vitamins

The composition of the present invention may comprise one or more oil-soluble vitamins. Examples of oil-soluble vitamins include, but are not limited to, oil-soluble versions of vitamin D, vitamin D derivatives, vitamin E (such as vitamin E acetate), vitamin E derivatives, pro-vitamins thereof, and mixtures thereof. The composition may comprise from about 0.0001% to about 50%, alternatively from about 0.001% to about 10%, alternatively from about 0.01% to about 8%, and alternatively from about 0.1% to about 5%, of the oil-soluble vitamin compound.

13. Other actives. The compositions of the present invention may comprise one or more of the following other actives or ingredients: fatty acids (especially poly-unsaturated fatty acids), glucosamine, zinc pyrithione (ZPT), anti-fungal agents, thiol compounds (e.g., N-acetyl cysteine, glutathione, thioglycolate), other vitamins, beta-carotene, ubiquinone, idebenone, amino acids, and hyaluronic acid increasing compounds/hyaluronidase inhibitors.

III. Dermatologically Acceptable Carrier

The personal care compositions of the present invention may comprise a dermatologically acceptable carrier. The dermatologically acceptable carrier may be present in an amount of from about 50% to about 99.99%, alternatively from about 60% to about 99.95%, alternatively from about 70% to about 98%, and alternatively from about 80% to about 95% by weight of the composition.

The carrier can be in a wide variety of forms. Non-limiting examples of emulsions useful herein include oil-in-water, water-in-oil, water-in-silicone, silicone in-water, water-in-oil-in-water, and oil-in-water-in-silicone emulsions. Alternatively, the emulsion is an oil-in-water emulsion. Emulsions also may contain a humectant, such as glycerin, and may contain from about 1% to about 10%, and alternatively from about 2% to about 5%, of a nonionic, anionic or cationic emulsifier. Examples of water-in-silicone and oil-in-water emulsions are described in U.S. Pat. No. 6,238,678, issued to Oblong et al.

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

The compositions of the present invention may also comprise other dermatologically acceptable topical carriers and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

V. Composition Forms

The personal care compositions of the present invention may be in a variety of forms, including but not limited to lotions, milks, mousses, serums, sprays, aerosols, foams, sticks, pencils, gels, creams and ointments, in-shower body lotions and/or body washes.

Compositions of this invention useful for cleansing (“cleansers”) may be formulated with a suitable carrier (e.g., as described above, and from about 1% to about 90%, by weight of the composition, of a dermatologically acceptable surfactant).

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

VI. Methods for Regulating Mammalian Keratinous Tissue

The compositions of the present invention are useful for regulating the condition of and/or beautifying mammalian keratinous tissue conditions by stimulating aquaporin membrane proteins. The present invention provides for a method for regulating the condition of mammalian skin. Regulating skin condition means improving skin appearance and/or feel, for example, providing a smoother, more even appearance and/or feel, as described further herein.

The method of regulating skin condition comprises the step of topically applying to the skin and/or other keratinous tissue an effective amount of a personal care composition of the present invention. Alternatively, the method of regulating skin condition comprises the step of applying a composition as described herein to mammalian skin which exhibits signs of skin aging and/or one or more conditions described herein that may be regulated and/or improved. The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The application of the present compositions may occur through a variety of means, non-limiting examples of which include using the palms of the hands and/or fingers, in combination with a delivery enhancement device, a temperature-change element, a substrate, an implement (e.g., a cotton ball, swab, pad, substrate, etc.), and combinations thereof. The method may comprise the step of inducing a temperature change in the composition either simultaneously or sequentially with the step of applying the composition to the keratinous tissue. Alternatively, the method may comprise the step of inducing a temperature change in the keratinous tissue either simultaneously or sequentially with application of the composition.

The present invention further may comprise a kit, said kit comprising an outer packaging unit, which in turn may comprise one or more smaller, inner packaging units. The smaller packaging units may comprise a unit dose of the composition, for example, about 0.1 ml to about 5.0 ml of composition. The kit further may comprise a plurality of components, including one or more compositions comprising an aquaporin-stimulating compound, one or more orally ingestible dietary supplements, a delivery enhancement device, an implement, a temperature-change element, a substrate, instructions for use of the device, instructions for complying with suitable application regimens, and combinations thereof. The compositions may be packaged in quantities suitable for use in a single application regimen, and alternatively in quantities suitable for multiple application regimens.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art.

### Examples 1-5

### Emulsions

| | Ex 1 | Ex 2 | Ex 3 | Ex 4 | Ex 5 |
|---|---|---|---|---|---|
| INGREDIENTS | % w/w | % w/w | % w/w | % w/w | % w/w |
| Deionised water | QS | QS | QS | QS | QS |
| Disodium EDTA | 0.1 | 0.1 | 0.1 | 0.1 | 0.1 |
| Glycerine | 10.0 | 15.0 | 25.0 | 20.0 | 20 |
| Cetearyl glucoside | 0.2 | 0.2 | 0.2 | 0.2 | 0.2 |
| Ginseng extract⁶ | 0.1 | — | — | 1 | — |
| Ginsenoside¹ | — | — | 1 | 0.01 | — |
| Caffeine | 5 | 1.5 | — | 1.5 | — |
| Pomegranate extract | — | — | — | — | 0.5 |
| Isohexadecane | 4.0 | 5.0 | 3.0 | 4.0 | 4.0 |
| Ethylparaben | 0.15 | 0.15 | 0.15 | 0.15 | 0.15 |
| Propylparaben | 0.07 | 0.07 | 0.07 | 0.07 | 0.07 |
| Stearic acid | 0.1 | 0.1 | 0.1 | 0.1 | 0.1 |
| PEG-100 Stearate | 0.1 | 0.1 | 0.1 | 0.1 | 0.1 |
| Stearyl alcohol | 0.6 | 0.6 | 0.6 | 0.6 | 0.6 |
| Cetyl alcohol | 0.5 | 0.5 | 0.5 | 0.5 | 0.5 |
| Behenyl alcohol | 0.4 | 0.3 | 0.2 | 0.2 | 0.2 |
| Isopropyl isostearate | 1.5 | 1 | 2 | 1.5 | 1.5 |
| DL- alpha Tocopherol | 0.25 | 0.50 | 0.25 | 0.50 | 0.5 |
| acetate | | | | | |
| Caprylic/Capric | 1.94 | — | — | 1.94 | 1.94 |
| Triglyceride and | | | | | |
| sodium acrylates | | | | | |
| copolymer² | | | | | |
| Mineral oil | 1.0 | 1.0 | 1.0 | — | — |
| Polyacrylamide & | | 2.5 | 2 | — | — |
| C13-14 Iso- | | | | | |
| paraffin & Laureth-7³ | | | | | |
| Glyceryl Polymethacry- | 0.5 | 1.0 | 0.5 | 0.1 | 1.0 |
| late and Propylene | | | | | |
| Glycol and PVM/MA | | | | | |
| Copolymer⁴ | | | | | |
| C12-13 Pareth 3 | 0.045 | — | — | 0.045 | 0.045 |
| Laureth-7 | 0.015 | — | — | 0.015 | 0.015 |
| Aluminium Starch | 1.0 | 0.5 | 0.5 | 0.5 | 1.0 |
| Octenyl succinate⁷ | | | | | |
| Polyethylene | 0.5 | 1.0 | 0.5 | 1.0 | 0.5 |
| homopolymer⁸ | | | | | |
| Niacinamide | 5.0 | 0.5 | 5.0 | — | 0.1 |
| D Panthenol | 1.0 | 0.5 | 1.0 | 1.0 | 1.0 |
| Sodium Hydroxide | 0.011 | 0.011 | 0.011 | 0.011 | 0.011 |
| Benzyl alcohol | 0.25 | 0.25 | 0.25 | 0.25 | 0.25 |
| Dimethicone & | 1.0 | 0.5 | 1.0 | 0.5 | 1.0 |
| Dimethiconol⁵ | | | | | |
| Perfume | 0.3 | 0.3 | 0.25 | 0.25 | 0.35 |

¹Ginsenoside is selected from Ginsenoside RB1, RG1 or RG3 - Wilshire Technologies - NJ - USA
²Luvigel ®- BASF
³Sepigel ® - Seppic
⁴Lubrajel ® - ISP
⁵DC1503 - Dow Corning ®
⁶Ginseng extract selected from Ginseng root extract or Ginseng flower extract
⁷Dry Flo ®Plus, National Starch, NJ, USA
⁸Microthene ®, Equistar Chemicals, Texas, USA

### Examples 6-11

### Gels

| | Ex 6 | Ex 7 | Ex 8 | Ex 9 | Ex 10 | Ex 11 |
|---|---|---|---|---|---|---|
| Components | % w/w | % w/w | % w/w | % w/w | % w/w | % w/w |
| Deionised Water | QS | QS | QS | QS | QS | QS |
| Disodium EDTA | 0.1 | 0.1 | 0.1 | 0.1 | 0.1 | 0.1 |
| Glycerine | 10.0 | 11.0 | 10.0 | 10.0 | 12.0 | 10 |
| Caffeine | 1.5 | 3.0 | 5.0 | — | 1.5 | 1.5 |
| Ginseng Extract⁷ | 0.1 | 0.1 | — | — | — | — |
| Pomegranate Extract⁶ | — | — | — | — | — | 0.5 |
| Ginsenoside⁵ | 0.5 | — | — | 1 | — | — |
| Niacinamide | 5.0 | 3.5 | 1.0 | 0.5 | — | 0.1 |
| D Panthenol | 1.0 | 0.5 | 1.0 | 0.5 | 1.0 | 1.0 |
| Dimethicone & Dimethiconol³ | 1.0 | 1.0 | 0.5 | 1.0 | 1.0 | 1.0 |
| Hexylene Glycol | 1.0 | 1.0 | 1.0 | 1.10 | 1.0 | 1.0 |
| Glyceryl Polymethacrylate and | 0.5 | 0.5 | 0.5 | 1 | 0.5 | 0.5 |
| Propylene Glycol and PVM/MA Copolymer⁴ | | | | | | |
| Caprylic/Capric Triglyceride And | 2.425 | — | — | — | 2.425 | 2.425 |
| Sodium Acrylates Copolymer¹ | | | | | | |
| C12-13 Pareth 3 | 0.051 | — | — | — | 0.051 | 0.051 |
| Xanthan gum | — | — | 0.2 | — | — | — |
| Laureth-7 | 0.024 | — | — | — | 0.024 | 0.024 |
| Alcohol | 5.0 | 3.0 | 5.0 | 3.0 | 5.0 | 3.0 |
| Polyacrylamide & C13-14 | — | 2.5 | 2.5 | 2.5 | — | — |
| Isoparaffin & Laureth 7² | | | | | | |
| Carbomer | — | — | 0.5 | — | — | — |
| Nylon 12 | 0.20 | 0.20 | 0.50 | 0.5 | 0.20 | 0.50 |
| Phenoxyethanol | 0.25 | 0.25 | 0.25 | 0.25 | 0.25 | 0.25 |
| Yellow No. 5 | 0.0024 | 0.0024 | 0.0024 | 0.0024 | 0.0024 | 0.0024 |
| FD&C Blue No 1 | 0.000205 | 0.000205 | 0.000205 | 0.000205 | 0.000205 | 0.000205 |
| Perfume | 0.3 | 0.3 | 0.3 | 0.25 | 0.3 | 0.3 |

¹Luvigel ® - BASF
²Sepigel ® - Seppic
³DC1503 - Dow Corning ®
⁴Lubrajel ® - ISP
⁵Ginsenoside is selected from Ginsenoside RB1, RG1 or RG3 - Wilshire Technologies - NJ, USA
⁶Alban Muller, Vincennes, France
⁷Selected from Ginseng Root Extract or Ginseng Flower Extract

### Examples 11-12

### Mousses

| | Ex 11 | Ex 12 |
|---|---|---|
| Components | % w/w | % w/w |
| Deionised water | QS | QS |
| Disodium EDTA | 0.1 | 0.1 |
| Glycerine | 10.0 | 12.0 |
| Polysorbate 20 | 1 | 1 |
| Cetearyl glucoside | 0.2 | 0.2 |
| Ginseng extract² | 0.1 | — |
| Ginsenoside¹ | — | 1 |
| Caffeine | 3 | 1.5 |
| Pomegranate extract | 0.5 | — |
| Isohexadecane | 3.0 | 3.0 |
| Ethylparaben | 0.15 | 0.15 |
| Propylparaben | 0.07 | 0.07 |
| Stearic acid | 0.1 | 0.1 |
| PEG-100 Stearate | 0.1 | 0.1 |
| Stearyl alcohol | 0.4 | 0.4 |
| Cetyl alcohol | 0.3 | 0.3 |
| Behenyl alcohol | 0.25 | 0.25 |
| Isopropyl isostearate | 1.5 | 1 |
| DL- alpha Tocopherol acetate | 0.25 | 0.50 |
| Citric acid | 0.01 | 0.01 |
| Mineral oil | 0.5 | 1.0 |
| Sodium chloride | 0.01 | 0.01 |
| Glyceryl Polymethacrylate and Propylene | 0.5 | 1.0 |
| Glycol and PVM/MA Copolymer⁴ | | |
| Acrylates/C10-30 alkylacrylate cross | 0.2 | 0.2 |
| polymer (Pemulen TR1)³ | | |
| Niacinamide | 5.0 | 3.0 |
| D Panthenol | 1.0 | 0.5 |
| Sodium Hydroxide | 0.05 | 0.05 |
| Benzyl alcohol | 0.25 | 0.25 |
| Dimethicone & Dimethiconol⁵ | 1.0 | 0.5 |
| Perfume | 0.3 | 0.3 |
| Propellant CAP40⁶ | 5 | 5 |

¹Ginsenoside is selected from Ginsenoside RB1, RG1 or RG3 - Wilshire Technologies - NJ - USA
²Ginseng extract selected from Ginseng Root extract or Ginseng Flower extract
³Noveon - OH- USA
⁴Lubrajel - ISP
⁵DC1503 - Dow Corning
⁶Propane/Iso-Butane/N-Butane (22:24:54) - Calor- Warwick - UK

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) from about 0.05% to about 5%, by weight of the entire composition, of at least one aquaporin-stimulating compound selected from the group consisting of xanthine, caffeine; 2-amino-6-methyl-mercaptopurine; 1-methyl xanthine; 2-aminopurine; theophylline; theobromine; adenine; adenosine; kinetin; p-chlorophenoxyacetic acid; 2,4-dichlorophenoxyacetic acid; indole-3-butyric acid; indole-3-acetic acid methyl ester; beta-naphthoxyacetic acid; 2,3,5-triiodobenzoic acid; adenine hemisulfate; n-benzyl-9-(2-tetrahydropyranyl)adenine; 1,3-diphenylurea; 1-phenyl-3-(1,2,3-thiadiazol-5-yl)urea; zeatin; indole-3-acetic acid; 6-benzylaminopurine; alpha-napthaleneacetic acid; 6-2-furoylaminopurine; green tea extract; white tea extract; menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor and combinations thereof;
b) an additional ingredient selected from the group consisting of niacinamide, glycerin and mixtures thereof; and
c) a dermatologically-acceptable carrier.

2. The composition of claim 1, further comprising at least one additional skin care active.

3. The composition of claim 1 wherein said aquaporin-stimulating compound and said glycerin are present in a weight percent ratio of from about 1 to about 6.66.

4. The composition of claim 1 comprising from about 9% to about 30% of glycerin.

5. The composition of claim 1 comprising from about 1% to about 8% of niacinamide.

6. The composition of claim 1, wherein the additional ingredient is glycerin.

7. The composition of claim 1, wherein the additional ingredient is niacinamide.

8. The composition according to claim 1, wherein the weight percent ratio of said aquaporin-stimulating compound, said glycerin and said niacinamide is about 1 to about 6 to about 3, respectively.

9. The method according to claim 1, wherein the weight percent ratio of said aquaporin-stimulating compound, said glycerin and said niacinamide is about 1 to about 4 to about 2, respectively.

10. The method according to claim 1, wherein the weight percent ratio of said aquaporin-stimulating compound, said glycerin and said niacinamide is about 1 to about 2 to about 1, respectively.

11. A method of regulating the condition of mammalian keratinous tissue comprising the step of applying to a portion of mammalian keratinous tissue in need of regulation a composition comprising:
a) from about 0.05% to about 5%, by weight of the entire composition, of at least one aquaporin-stimulating compound selected from the group consisting of xanthine, caffeine; 2-amino-6-methyl-mercaptopurine; 1-methyl xanthine; 2-aminopurine; theophylline; theobromine; adenine; adenosine; kinetin; p-chlorophenoxyacetic acid; 2,4-dichlorophenoxyacetic acid; indole-3-butyric acid; indole-3-acetic acid methyl ester; beta-naphthoxyacetic acid; 2,3,5-triiodobenzoic acid; adenine hemisulfate; n-benzyl-9-(2-tetrahydropyranyl)adenine; 1,3-diphenylurea; 1-phenyl-3-(1,2,3-thiadiazol-5-yl)urea; zeatin; indole-3-acetic acid; 6-benzylaminopurine; alpha-napthaleneacetic acid; 6-2-furoylaminopurine; green tea extract; white tea extract; menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor and combinations thereof;
b) an additional ingredient selected from the group consisting of niacinamide, glycerin and mixtures thereof; and
c) a dermatologically-acceptable carrier.

12. The method according to claim 11, wherein the composition further comprises at least one additional skin care active.

13. The method according to claim 11, wherein said aquaporin-stimulating compound and said glycerin are present in a weight percent ratio of from about 1 to about 6.66.

14. The method according to claim 11 comprising from about 9% to about 30% of glycerin.

15. The composition of claim 11, comprising from about 1% to about 8% of niacinamide.

16. The method according to claim 11 wherein the additional ingredient is glycerin.

17. The method according to claim 11, wherein the additional ingredient is niacinamide.

18. The method according to claim 11, wherein the weight percent ratio of said aquaporin-stimulating compound, said glycerin and said niacinamide is about 1 to about 6 to about 3, respectively.

19. The method according to claim 11, wherein the weight percent ratio of said aquaporin-stimulating compound, said glycerin and said niacinamide is about 1 to about 4 to about 2, respectively.

20. The method according to claim 11, wherein the weight percent ratio of said aquaporin-stimulating compound, said glycerin and said niacinamide is about 1 to about 2 to about 1, respectively.

21. A kit comprising:
a) a composition comprising:
i. from about 0.05% to about 5%, by weight of the entire composition, of at least one aquaporin-stimulating compound selected from the group consisting of xanthine, caffeine; 2-amino-6-methyl-mercaptopurine; 1-methyl xanthine; 2-aminopurine; theophylline; theobromine; adenine; adenosine; kinetin; p-chlorophenoxyacetic acid; 2,4-dichlorophenoxyacetic acid; indole-3-butyric acid; indole-3-acetic acid methyl ester; beta-naphthoxyacetic acid; 2,3,5-triiodobenzoic acid; adenine hemisulfate; n-benzyl-9-(2-tetrahydropyranyl)adenine; 1,3-diphenylurea; 1-phenyl-3-(1,2,3-thiadiazol-5-yl)urea; zeatin; indole-3-acetic acid; 6-benzylaminopurine; alpha-napthaleneacetic acid; 6-2-furoylaminopurine; green tea extract; white tea extract; menthol; tea tree oil; ginsenoside-RB1; ginsenoside-RB3; ginsenoside-RC; ginsenoside-RD; ginsenoside-RE; ginsenoside-RG1; ginseng root extract; ginseng flower extract; pomegranate extract, extracts from Ajuga turkestanica; extracts from viola tricolor and combinations thereof;
ii. an additional ingredient selected from the group consisting of niacinamide, glycerin and mixtures thereof;
iii. a dermatologically-acceptable carrier; and
b) at least one additional component selected from the group consisting of an orally ingestible dietary supplement, a delivery enhancement device, an implement, a temperature-change element, a substrate, instructions for use of the device, instructions for complying with suitable application regimens, and combinations thereof.
